(19) United States
(12) Patent Application Publication
(10) Pub. No.: US 2006/0127486 A1
(43) Pub. Date: Jun. 15, 2006

(54) Ceramic structures for prevention of drug diversion

(76) Inventors: Rudi E. Moerck, San Antonio, TX (US); Bruce J. Sabacky, Reno, NV (US); Timothy M. Spitler, Fernley, NV (US); Jan Prochazka, Reno, NV (US); Douglas Ellsworth, Reno, NV (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/181,667

(22) Filed: Jul. 13, 2005

Related U.S. Application Data
(60) Provisional application No. 60/587,662, filed on Jul. 13, 2004.

(51) Int. Cl. A61K 31/485; A61K 9/14

(52) U.S. Cl. 424/489; 514/282; 977/906

(57) ABSTRACT

The present invention is directed to compositions that provide drug delivery while resisting methods of diversion. The compositions are combinations of the drug and a ceramic structure. Any suitable drug may be used, but the drug is typically an opioid agonist. The ceramic structures arc usually metal oxides, and are oftentimes roughly spherical in shape with a hollow center.

### CROSS REFERENCE TO RELATED APPLICATIONS

This application claims priority to U.S. provisional patent application No. 60/587,662, the entire disclosure of which is incorporated by reference.

### FIELD OF INVENTION

The present invention generally relates to the prevention of drug diversion. More specifically, it relates to drug/ceramic structure combinations that provide drug delivery while resisting methods of diversion.

### BACKGROUND OF INVENTION

Drug diversion is the use of a prescribed medication by a person for whom the medication was not prescribed. Such use accounts for almost 30% of drug abuse in the United States and represents a close challenge to cocaine addiction. The majority of abusers are persons with no history of prior drug abuse who became addicted after using prescription drugs for legitimate medical reasons.

It is well-known that abusers of prescribed medication target two parameters when diverting drugs—dose amount and dose form for rapid administration. A diverter will oftentimes obtain a drug, crush it, and then deliver it intranasally. Another mode of administration involves dissolving the drug in water or alcohol and then delivering it intravenously. Either delivery mode provides for rapid drug introduction into the bloodstream.

Several methods have been developed to inhibit drug diversion. One such method involved the incorporation of the target drug into a polymer matrix. The idea was to adsorb drug within the polymer matrix, which would only allow its slow release upon introduction to a solvent. In other words, one could not directly access the incorporated drug, even through an extraction process. This strategy ultimately failed, however, when diverters discovered that they could simply crush the polymer matrix, which provided ready access to the adsorbed drug.

There is accordingly a need for a novel method for inhibiting or preventing drug diversion. That is an object of the present invention.

### SUMMARY OF INVENTION

The present invention is directed to drug/ceramic structure combinations that provide drug delivery while resisting methods of diversion. The ceramic structure typically includes a metal oxide, wherein the oxide is of titanium, zirconium, scandium, cerium, or yttrium. Any suitable drug may be used in the combinations, but opioid agonists are preferred, especially oxycodone.

In a composition aspect of the present invention, a composition comprising a ceramic structure and a drug is provided. The ceramic structure is roughly spherical and hollow. The drug is coated in the hollow portion of the ceramic structure, and the mean diameter of the structure ranges from 10 nm to 100 μm. The mean particle diameter oftentimes ranges according to the following: 10 nm to 100 nm; 101 nm to 200 nm; 201 nm to 300 nm; 301 nm to 400 nm; 401 nm to 500 nm; 501 nm to 600 nm; 601 nm to 700 nm; 701 nm to 800 nm; 801 nm to 900 nm; 901 mm to 1 μm; 1 μm to 10 μm; 11 μm to 25 μm; and, 26 μm to 100 μm. Variation in particle size is typically less than 10.0% of the mean diameter, preferably less than 7.5% of the mean diameter, and more preferably less than 5.0% of the mean diameter.

The ceramic structure typically includes titanium oxide or zirconium oxide. The included drug is typically an opioid agonist selected from oxycodone, codeine, hydrocodone, hydromorphone, levorphanol, meperidine, methandone, and morphine. Ceramic structure/drug combinations of the present invention exhibit measurable mechanical strength. At least 50 percent of the particles maintain their overall integrity (e.g., shape, size, porosity, etc.) when a force of 5 kg/cm², 7.5 kg/cm², 10.0 kg/cm², 12.5 kg/cm², 15.0 kg/cm², 17.5 kg/cm² or 20 kg/cm² is applied to them.

### DETAILED DESCRIPTION

The present invention is directed to drug/ceramic structure combinations that provide drug delivery while resisting methods of diversion.

One can incorporate any suitable drug into the combination of the present invention, although opioid agonists are preferred. Such agonists include, without limitation, the following: alfentanil, allylprodine, alphaprodine, anileridine, benzylmorphine, bezitramide, buprenoiphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiamhutene, ethylmorphine, etonitazene, etorphine, dihydroetorphine, fentanyl, hydrocodone, hydromorphone, hydroxypethidine, isomethadone, ketobemidone, levorphanol, levophena.cylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, pharmaceutically acceptable salts thereof, stereoisomers thereof, ethers thereof, esters thereof, and mixtures thereof.

Examples of other drugs that may be incorporated into ceramic structures include, without limitation, the, following: acetorphine, alphacetylmethadol, alphameprodine, alphamethadol, alphaprodine, aenzethidine, betacetylmethadol, betameprodine, betamethadol, betaprodine, bufotenine, carfentanil, diamorphine, diethylthiambutene, difenoxin, dihydrocodeinone, drotebanol, eticyclidine, etoxeridine, etryptanrine, furethidine, hydromoiphinol, levomethorphan, levomoramide, methadyl acetate, methyldesorphin, methyldihydroniorphine, morpheridine, noracymethadol, pethidine, phenadoxone, phenampromide, phencyclidine, psil.ocin, racemethorphan, racemoramide, racemorphan, rolicyclidine, tenocyclidine, thebacon, thebaine, tilidate, trimeperidine, acetyldihydrocodeine, amphetamine, glutethimide, lefetamine, mecloqualone, methaqualone, methcathinone, methylamphetamine, methylphenidate, methylphenobarbitone, nicocodine, nicodicodinc, norcodeine, phenmetrazine, pholcodine, propiram, zipeprol, alprazolam, a minorex, benzphetamine, bromazepam., brotizolam, camazepam, cathine, cathinone, ehlordiazepoxide, chlorphentermine, clobazam, elonazepam, clorazepic acid, clotiazepam, cloxazolam, delorazepam, dextropropoxyphene, diazepam, diethylpropion, estazolarn, ethchlorvynol, ethinamate, ethyl loflazepate, fencamfamin, fenethylline, fenproporex, fludiazepam, flunitrazepam, flurazepam, halazepam, haloxazolam, ketazolam, loprazolam, lorazepam, lormetazepam, mazindol, medazepam, mefenorex, mephentermine, meprobamate, mesocarb, methyprylone, midazolam, nimetaz.epam, nitrazepam, nordazepam, oxazepam, oxazolam, pemoline, phendimetrazine, phentermine, pinazeparn, pipadrol, prazeparn, pyrovalerone, temazepam, tetrazepam, triazolam, N-ethylamphetamine, atamestane, bolandiol, bolasterone, bolazine, boldenone, bolenol, bolmantalate, calusterone, 4-chloromethandienone, clostebol, drostanolone, enestebol, epitiostanol, ethyloestrenol, fluoxymesterone, formebolone, furazabol, mebolazine, mepitiostane, mesabolone, mestarolone, mesterolone, methandienone, methandriol, methenolone, metribolone, mibolerone, nandrolone, norboletone, norclostebol, norethandrolone, ovandrotone, oxabolone, oxandrolone, oxymesterone, oxymetholone, prasterone, propetandrol, quinbolone, roxibolone, silandrone, stanolone, stanozolo, stenbolone, pharmaceutically acceptable salts thereof, stereoisomers thereof, ethers thereof, esters thereof, and mixtures thereof.

Ceramic structures of the present invention typically include oxides of titanium, zirconium, scandium, cerium, and yttrium, either individually or as mixtures. Preferably, the ceramic is a titanium oxide or a zirconium oxide, with titanium oxides being especially preferred. Structural characteristics of the ceramics are well-controlled, either by synthetic methods or separation techniques. Examples of controllable characteristics include: 1) whether the structure is roughly spherical and hollow or a collection of smaller particles bound together in approximately spherical shapes; 2) the range of structure sizes (e.g., particle diameters); 3) surface area of the structures; 4) wall thickness, where the structure is hollow; 5) pore size range; and, 6) strength of structural integrity.

The ceramics are typically produced by spray hydrolyzing a solution of a metal salt to form particles, which are collected and heat treated. Spray hydrolysis initially affords noncrystalline hollow spheres. The surface of the spheres consists of an amorphous, glass-like film of metal oxide or mixed-metal oxides. Calcination, or heat treatment, of the material causes the film to crystallize, forming an interlocked framework of crystallites. The calcination products are typically hollow, porous, rigid structures.

A variety of roughly spherical ceramic materials are produced through the variation of certain parameters: a) varying the metal composition or mix of the original solution; b) varying the solution concentration; and, c) varying calcination conditions. Furthermore, the materials can be classified according to size using well-known air classification and sieving techniques.

In the case of roughly spherical, hollow structures, particles sizes typically range from 10 nm to 100 μm. The mean particle diameter oftentimes ranges according to the following: 10 nm to 100 nm; 101 nm to 200 nm; 201 nm to 300 nm; 301 nm to 400 nm; 401 nm to 500 nm; 501 nm to 600 nm; 601 nm to 700 nm; 701 nm to 800 nm; 801 nm to 900 nm; 901 nm to 1 μm; 1 μm to 10 μm; 11 μm to 25 μm; and, 26 μm to 100 μm.

Variation in particle size throughout a sample is typically well-controlled. For instance, variation is typically less than 10.0% of the mean diameter, preferably less than 7.5% of the mean diameter, and more preferably less than 5.0% of the mean diameter.

Surface area of the ceramic structures depends on several factors, including particle shape, particle size, and particle porosity. Typically, the surface area of roughly spherical particles ranges from 0.1 m²/g to 100 m²/g. The surface area oftentimes, however, ranges from 0.5 m²/g to 50 m²/g.

Wall thicknesses of hollow particles tend to range from 10 nm to 5 μm, with a range of 50 nm to 3 μm being typical. Pore sizes of such particles further range from 1 nm to 5 μm, and oftentimes lie in the 5 nm to 3 μm range.

The ceramic structures of the present invention exhibit substantial mechanical strength. At least 50 percent of the particles maintain their overall integrity (e.g., shape, size, porosity, etc.) when a force of 5 kg-force/cm² (45 newtons/cm²), 7.5 kg-force/cm² (67.5 newtons/cm²), 10.0 kg-force/cm² (90 newtons/cm²), 12.5 kg-force/cm² (112.5 newtons/cm²), 15.0 kg-force/cm² (135 newtons/cm²), 17.5 kg-force/cm² (157.5 newtons/cm²), 20 kg-force/cm² (180 newtons/cm²), 35 kg-force/cm² (315 newtons/cm²), 50 kg-force/cm² (450 newtons/cm²), 75 kg-force/cm² (675 newtons/cm²), 100 kg-force/cm² (900 newtons/cm²), or even 125 kg-force/cm² (1125 newtons/cm²) is applied to them. Typically, at least 60 percent of the particles maintain their integrity. Preferably, at least 70 percent of the particles maintain their integrity, with at least 80 percent being more preferred and at least 90 percent being especially preferred.

Without further treatment, the ceramic structures of the present invention are hydrophilic. The degree of hydrophilicity, however, may be chemically modified using known techniques. Such techniques include, without limitation, treating the structures with salts or hydroxides containing magnesium, aluminum, silicon, silver, zinc, phosphorous, manganese, barium, lanthanum, calcium, cerium, and PEG polyether or crown ether structures. Such treatments influence the ability of the structures to uptake and incorporate drugs, particularly hydrophilic drugs, within their hollow space.

Alternatively, the structures may be made relatively hydrophobic through treatment with suitable types of chemical agents. Hydrophobic agents include, without limitation, organo-silanes, chloro-organo-silanes, organo-alkoxy-silanes, organic polymers, and alkylating agents. These treatments make the structures more suitable for the incorporation of lipophilic or hydrophobic drugs. Additionally, the porous, hollow structures may be treated using chemical vapor deposition, metal vapor deposition, metal oxide vapor deposition, or carbon vapor deposition to modify their surface properties.

The drug that is applied to the ceramic structures may optionally include an excipient. Examples of excipients include, without limitation, the following: acetyltriethyl citrate; acetyltrin-n-butyl citrate; aspartame; aspartame and lactose; alginates; calcium carbonate; carbopol; carrageenan; cellulose; cellulose and lactose combinations; croscarmellose sodium; crospovidone; dextrose; dibutyl sehacate; fructose; gellan gum., glyceryl behenate; magnesium stearate; maltodextrin; maltose; mannatol; carboxymethylcellulose; polyvinyl acetate phathalate; povidone; sodium starch glycolate; sorbitol; starch; sucrose; triacetin; triethyleitrate; and, xanthan gum.

A drug may be combined with a ceramic structure of the present invention using any suitable method, although solvent application/evaporation and drug melt are preferred. For solvent application/evaporation, a drug of choice is dissolved in an appropriate solvent. Such solvents include, without limitation, the following: water, buffered water, an alcohol, esters, ethers, chlorinated solvents, oxygenated solvents, organo-amines, amino acids, liquid sugars, mixtures of sugars, supercritical liquid fluids or gases (e.g., carbon dioxide), hydrocarbons, polyoxygenated solvents, naturally occurring or derived fluids and solvents, aromatic solvents, polyaromatic solvents, liquid ion exchange resins, and other organic solvents. The dissolved drug is mixed with the porous, hollow ceramic structures, and the resulting suspension is degassed using pressure swing techniques or ultrasonics. While stirring the suspension, solvent evaporation is conducted using an appropriate method (e.g., vacuum, spray drying under low partial pressure or atmospheric pressure, and freeze drying).

Alternatively, the above-described suspension is filtered, and the coated ceramic particles are optionally washed with a solvent. The collected particles are dried according to standard methods. Another alternative involves filtering the suspension and drying the wet cake using techniques such as vacuum drying, air stream drying, microwave drying and freeze-drying.

For the drug melt coating method, a melt of the desired drug is mixed with the porous, hollow ceramic structures under low partial pressure conditions (i.e., degassing conditions). The mix is allowed to equilibrate to atmospheric pressure and to cool under agitation. This process affords a powder with drug both inside and outside the structures. Drug may be removed from the particle surface prior to tableting by simple washing of the particle surface with an appropriate solvent and subsequent drying.

Drug on the inside of the ceramic structures is typically coated in a thickness ranging from 10 nm to 10 μm, with 50 nm to 5 μm being preferred. The corresponding weight ratio of drug to particle usually ranges from 1.0 to 100, with a range of 2.0 to 50 being preferred.

Coated drug may exist in either a crystalline or amorphous (noncrystalline) form. Crystalline materials exhibit characteristic shapes and cleavage planes due to the arrangement of their atoms, ions or molecules, which form a definite pattern called a lattice. An amorphous material does not have a molecular lattice structure. This distinction is observed in powder diffraction studies of materials: In powder diffraction studies of crystalline materials, peak broadening begins at a grain size of about 500 nm. This broadening continues as the crystalline material gets small until the peak disappears at about 5 nm By definition, a material is “amorphous” by XRD when the peaks broaden to the point that they are not distinguishable from background noise, which occurs at 5 nm or smaller.

The coated drug on the particle is in a substantially pure form. Typically, the drug is at least 95.0% pure, with a purity value of at least 97.5% being preferred and a value of at least 99.5% being especially preferred. In other words, drug degradants (e.g., hydrolysis products, oxidation products, photochemical degradation products, etc.) are kept below 0.5%, 2.5% or 5.0% respectively.

The drug/ceramic structure combination of the present invention provides for drug delivery when administered by a variety of methods, typically through oral administration. Typically, the combination provides for the release of at least 25 percent of the included drug, preferably at least 50 percent of the included drug, and more preferably at least 75 percent of the included drug.

The drug/ceramic structure combination of the present invention, when administered to a patient, typically provides for controlled delivery of the drug to the patient. Usually, when the subject combination is tested using the LISP Paddle Method at 100 rpm in 900 ml aqueous buffer (pH between 1.6 and 7.2) at 37° C., the following dissolution profile will be provided: between. 5.0% and 50.0% of the drug released after 1 hour; between 10.0% and 75.0% of the drug released after 2 hours; between 20.0% and 85.0% of the drug released after 4 hours; and, between 25.0% and 95.0% of the drug released after 6 hours. Oftentimes, from hour I until hour 4, 5 or 6, drug release is observed to follow zero-order kinetics.

Drug/ceramic structure combinations of the present invention are particularly resistant to diversion attempts. As note above, the ceramic structures exhibit substantial mechanical strength, which affords integrity to the combination as well. Typically, when the combinations are subjected to a force of 5.0, 7.5, 10.0, 12.5, 15.0, 17.5 or 20.0 kg/cm², and then tested using the USP Paddle Method described above, the ratio of dissolution rate post-force application to pre-force application is less than 2.0. Preferably it is less than 1.7, more preferably less than 1.5, and most preferably less than 1.3.

Typically, when opioid agonists are used in the combination of the present invention, from 75 ng to 750 mg of the agonist is included. The exact amount will depend on the particular opioid agonist and can be determined using well-known methods. Studies have furthermore been performed outlining equianalgesic doses of various opioids, which can aid in the exact dose determination, including the following: oxycodone (13.5 mg); codeine (90.0 mg); hydrocodone (15.0 mg); hydromorphone (3.375 mg); levorphanol (1.8 mg); meperidine (135.0 mg); methadone (9.0 mg); and, morphine (27.0 mg).

The opioid agonist dose may be optionally reduced through inclusion of an additional non-opioid agonist, such as an NSAID or a COX-2 inhibitor. Examples of NSAIDs include, without limitation, the following: ibuprofen; diclofenac; naproxen; benoxaprofen; flurbiprofen; fenoprofen; flubufen; ketoprofen; inodoprofen; piroprofen; carprofen; oxaprozin; pramoprofen; muroprofen; trioxaprofen; suprofen; aminoporfen; tiaprofenic acid; fluprofen; bucloxic acid; indomethacin; sulindac; tolmetin; zomepirac; tiopinac; zidometacin; acemetacin; fentiazac; clidanac; oxpinac; mefenamic acid; meclofenamic acid; flufenamic acid; niflumic acid; tolfenamic acid; diflurisal; flufenisal; piroxicam; sudoxicam; and isoxicam. COX-2 inhibitors include, without limitation, celecoxib, flosulide, moloxicam, 6-methoxy-2 naphtylacetic acid, vioxx, nabumetone, and nimesulide. Useful dosages of the preceding NSAIDs and COX-2 inhibitors are well-known in the art.

The drug/ceramic structure combinations exhibit beneficial stability characteristics under a number of conditions. In other words, the included drug does not substantially decompose over reasonable periods of time. At 25° C. over a two week period for instance, the drug purity typically degrades less than 5%. Oftentimes, there is less than 4%, 3%, 2%, or 1% degradation (e.g., hydrolysis, oxidation, photochemical reactions).

The following examples are meant to illustrate the present invention and are not meant to limit it in any way.

### EXAMPLE 1

An aqueous solution of titanium oxychloride and HCl containing 15 g/l Ti and 55 g/l Cl was injected in a titanium spray drier at a rate of 12 liters/h. The outlet temperature from the spray drier was 250° C. A solid intermediate product consisting of amorphous spheres was recovered on a bag filter. The inteiniediate product was calcined in a muffle furnace at 500° C. for 8 h. The calcined material was further classified by passing it through a set of cyclones. The size fraction 15-25 μm was screened to eliminate any particles not present as spheres. X-Ray diffraction shows that product is made primarily of TiO2 rutile, with about 1% anatase. The average mechanical strength of the particles was measured by placing a counted number of them on a flat metal surface, positioning another metal plate on top and progressively applying pressure until the particles begin to break. Scanning electron micrographs of the calcined product show that it is made of rutile crystals, bound together as a thin-film structure. The thickness of the film is about 500 nm and the pores have a size of about 50 n.

### EXAMPLE II

The experiment of example I was repeated at different temperatures over the range 500 to 900° C., with different concentrations of chloride and titanium in solution and with different nozzle sizes. The titanium concentration was varied over the range 120 to 15 g/l Ti. In general, a higher temperature creates larger and stronger particles, a lower Ti concentration tends to decrease the size of the spheres, to increase the thickness of the walls and to increase the mechanical strength of the particles.

### EXAMPLE III

The conditions were the same as those of Example I, except that a eutectic mixture of chloride salts of Li, Na and K equivalent to 25% of the amount of TiO2 present was added to the solution before the spraying step and a washing step was added after the calcination step. In the washing step, the calcined product was washed in water and the alkali salts were thereby removed from the final product. The advantage of using the salt addition is that the spheres of the final product have a thicker wall.

### EXAMPLE IV

The conditions were the same as those of Example I, except that an amount of sodium phosphate Na₃PO₄ equivalent to 3% of the amount of TiO2 present was added to the solution before spraying. The additive ensured faster rutilization of the product during calcination. The final product produced in this example consisted of larger rutile crystals than in the other examples, and exhibited a higher mechanical strength.

### EXAMPLE V

The product of Example I was slurried in water to make a slurry containing 40% solids. An amount of silver in colloidal form, corresponding to 5 weight % of the amount of TiO2 present was added to the slurry. The slurry with the colloidal silver added was injected in a spray drier with an outlet temperature of 250° C. and recovered on a bag filter. The intermediate product recovered on the bag filter was further calcined in a muffle furnace for 3 h at 600° C. Scanning electron micrography shows that the final product consists of hollow spheres with an average diameter of 50 μm, made of bound rutile crystals of about 2 μm in size. The pore size was about 500 nm. The colloidal silver forms a layer about 2 nm thick on the surface of the particles of the structure.

### EXAMPLE VI

Example V was repeated in different conditions of temperature and concentration and with different compounds serving as ligands. The following compounds were used as ligands: proteins, enzymes; polymers; colloidal metals, metal oxides and salts; active pharmaceutical ingredients. Temperatures are adapted to take into account the stability of the ligands. With organic compounds, the temperature is generally limited to about 150° C.

### EXAMPLE VII

A 10 ml vial of latex (Polysciences 0.5 μm microspheres at 2.5 wt % in 10 mL water) was diluted to a total volume of 40 mL with distilled water. The resulting mixture was treated with 0.36 g Tyzor LA® (DuPont). The latex/Tyzor LA® mixture was continuously stirred with a stir bar. About 0.5 mL/hour of acid was metered into the mixture using peristaltic pumps. pH was continuously monitored and values were recorded over time. The mixture's pH was titrated to pH 2. The latex was dip coated onto substrate, and the organic latex was removed by oxidation at 600° C. Variation in the approximately 0.5 μm diameter, hollow ceramic particles was typically less than 5.0% of the mean diameter. By using smaller microspheres, this process can produce substantially smaller particles (e.g., 0.1 μm, 0.05 μm and 0.02 μm) with similar uniformity.

1. A composition comprising a ceramic structure and a drug, wherein the ceramic structure is roughly spherical and hollow, and wherein the drug is coated in the hollow portion of the ceramic structure, and wherein the mean structure diameter ranges from 10 nm to 100 μm.

2. The composition of claim 1, wherein the ceramic structure comprises an oxide, and wherein the oxide is selected from a group consisting of titanium, zirconium, scandium, cerium, yttrium and mixtures thereof.

3. The composition of claim 2, wherein the ceramic structure comprises a titanium oxide or a zirconium oxide.

4. The composition of claim 3, wherein the ceramic structure comprises a titanium oxide.

5. The composition of claim 1, wherein the mean structure diameter ranges from 10 nm to 1 μm.

6. The composition of claim 5, wherein the structure diameter ranges from 5 μm to 25 μm.

7. The composition of claim 1, wherein the coated drug is an opioid agonist.

8. The composition of claim 7, wherein the opioid agonist is selected from a group consisting of oxycodone, codeine, hydrocodone, hydromorphone, levorphanol, meperidine, methadone, and, morphine.

9. The composition of claim 8, wherein the opioid agonist is oxycodone or morphine.

10. The composition of claim 1, wherein the ceramic structure comprises pores, and wherein the pore sizes range from 1 nm to 5 μm.

11. The composition of claim 10, wherein the ceramic structure comprises pores, and wherein the pore sizes range from 5 nm to 3 μm.

12. The composition of claim 1, wherein the hollow ceramic structure has a wall thickness, and wherein the thickness ranges from 10 nn to 5 μm.

13. The composition of claim 12, wherein the wall thickness ranges from 50 nm to 3 μm.

14. The composition of claim 1, wherein the ceramic structure exhibits a measurable mechanical strength, and wherein the mechanical strength is expressed in terms of a collection of particles, and wherein at least 50 percent of the particles maintain their overall integrity when a force of 5 kg/cm2 is applied to them.

15. The composition of claim 14, wherein at least 70 percent of the particles maintain their overall integrity.

16. The composition of claim 15, wherein at least 90 percent of the particles maintain their overall integrity.

17. The composition of claim 16, wherein a force of 10.0 kg/cm² is applied.

18. The composition of claim 17, wherein a force of 15.0 kg/cm² is applied.
